(19) United States
(12) Patent Application Publication
(10) Pub. No.: US 2012/0197160 A1
(43) Pub. Date: Aug. 2, 2012

(54) DEVICE FOR DETECTING AND/OR INFLUENCING POSTURE

(75) Inventors: Holger Reinhardt, Kempen (DE); Olaf Kroll-Orywahl, Gottingen (DE)

(73) Assignee: OTTO BOCK HEALTHCARE GMBH, Duderstadt (DE)

(21) Appl. No.: 13/501,226

(22) PCT Filed: Sep. 9, 2010
(86) PCT No.: PCT/DE10/01071

§ 371 Date: Apr. 10, 2012

(30) Foreign Application Priority Data
Oct. 22, 2009 (DE) 10 2009 050 385.4

(51) Int. Cl. A61B 5/103

(52) U.S. Cl. 600/587

(57) ABSTRACT

The invention relates to a device (1) for detecting and/or influencing posture, comprising a flexible support element (2), on which a fixing unit (3) for arranging the device (1) at the waist height of a wearer of the device (1) is arranged. First ends (41, 51) of the tension straps (4, 5) are fastened to the fixing unit (3) and guided so as to cross each other in the back area, second ends (42, 52) of the tension straps being fastened to the support element (2) in the frontal shoulder or chest area.

The invention relates to a device for detecting and/or influencing posture, having a flexible support element, on which a fixing apparatus is arranged for arranging the device at waist height of a wearer of the device, or below. Such a device serves, in particular, for analyzing movement or for supporting the posture in the region of the torso.

DE 10 2005 058 850 A1 describes an applicator for independent TENS treatment on the body with a suspender-like strap system that can be secured to the waistband of a pair of trousers using detachable clamps. Displaceable electrodes are attached to the strap system. The strap system has two longitudinal bands that are spanned inward via spacer bands and connected to the latter by double clasps.

U.S. Pat. No. 5,143,088 describes a device for monitoring the movement components of the spine. An elongate exoskeleton with a multiplicity of substantially flat, T-shaped elements which are spaced apart in the longitudinal direction is attached to the back of a person. The attachment is brought about by a hip strap and shoulder straps that protrude laterally from the upper end of the exoskeleton. The device is secured on the body by touch-and-close fasteners.

DE 20 2008 003 075 U1 describes an elastic cable system for supporting physiological posture. The elastic cable system can be placed on the upper body of a person and provides a flexible rubber cable that is connected to form a cable system by two spring clips and can be controlled in its length by a cord lock. Bad posture becomes perceivable by pressure in the region of the clavicles. Fixing in the region of the lumbar spine is brought about by a strap loop.

WO 2006/121413 A1 describes a vest that should improve posture. A waist strap is provided with an elastic back tape. Elastic strips that are attached to non-elastic strips or straps are arranged on the elastic back tape in the region of the shoulders. The non-elastic straps in turn are secured to the lower end of the back tape such that they are adjustable in length.

U.S. Pat. No. 6,440,094 B1 describes a device for improving the posture. In one embodiment, two tapes are guided so as to cross each other in the region of the back of a user, respectively by means of a tube element, wherein each tape can be fixed to a shoulder element with one end and, laterally to the left or right of the user, to a waist strap with another end. These features are acknowledged in similar form in the preamble of the claim.

An object of the present invention is to make available a device that enables improved movement analysis and supplies improved feedback in respect of the movement behavior.

According to the invention, this object is achieved by a device having the features of the main claim. Advantageous embodiments and developments of the invention are listed in the dependent claims.

The device according to the invention for detecting and/or influencing posture, having a flexible support element, on which a fixing apparatus is arranged for arranging the device at waist height or below, with tension straps or pull elements being fixed on the fixing apparatus with a first end and guided so as to cross each other in the region of the back and being fixed to the support element in the frontal shoulder or chest region with a second end, provides for at least one sensor to be associated with the support element, the tension straps and/or stiffening elements and for it to detect e.g. forces, deformations, angles and/or accelerations. As a result, it is possible to combine the device with a sensor system for detecting various movement states, for documenting these and optionally for evaluating these. By way of example, the sensors can be embodied as conductive textile threads, gyroscopes, accelerometers, extensometers, pressure sensors or temperature sensors. The angle can be measured in absolute terms or relative to a component or a body part.

Guiding the tension straps so as to cross in particular affords the possibility of detecting the torsional movements of the torso, which, in principle, are critical, and of providing feedback, by exerting a tensile force or compressive force on the respective side twisted forward, that the torso is currently in a twisted position. Provided that the wearer of the device bends forward uniformly, the uniform bending is indicated by the exertion of pull and pressure in the shoulder or chest region, and so the wearer of the device is encouraged to have an erect posture or at least is made aware of the position and posture he finds himself in.

Attachment apparatuses can be arranged on the support element in the frontal shoulder or chest region and the second ends of the tension straps can be reversibly fixed to these. As a result, the exerted counterforce can be set in a continuously variable manner by the tension straps. The respectively wanted posture can be set in a continuously variable manner as a result of this, and so a fit to the respectively wanted posture is easy to come by. Arranging the attachment apparatuses in the chest or shoulder region makes setting easier because it is easy to reach these body regions. The first ends, which are fixed to the fixing apparatus in the region of the waist, are preferably fixed in a permanent manner, for example by being sewn or welded together; alternatively, these ends can also be reversibly fixed to the fixing apparatus in the region of the waist, for example by hook-and-loop fasteners or other interlocking connections such as clasps, buttons or the like.

The support element is preferably embodied as a jacket or vest, which is easy to put on. The jacket or vest can be opened in the front region, for example by a zip fastener or a hook-and-loop fastener, and can just as easily be closed again such that the support element lies securely against the body. In the region of the back, the support element can have a design stretching over the whole area and completely cover the back; however, it is also possible for the support element to have through-holes such that the region of the back and the frontal region are only partly covered by the support element. Here, the support element can be worn directly on the skin over appropriate clothes.

The fixing apparatus for fixing the device in the region of waist height, or below this, is preferably embodied as a tension strap which can be part of the support element. The tension strap or waist strap can be secured to, or formed on, the remaining part of the support element, for example at the lower end of the vest or jacket, so as to form an integral component of the support apparatus. This simplifies putting on the device because it is only necessary for the jacket or vest to be put on and the tension strap to be tensioned in order to exert a sufficient peripheral force so that the fixing apparatus is securely held on the body. As an alternative to this, provision is made for the fixing apparatus to be embodied as separate components that can be coupled to one another such that, for example, the fixing apparatus can be put on independently of putting on the support element.

In addition to the embodiment of the fixing apparatus as tension strap, which is put on around the torso, it is possible that it is embodied as a pair of trousers or as a strap in the crotch. Here, the goal of the fixing apparatus is to distally fix the support element in order to prevent it from sliding up.

A lumbar support can be arranged in the support element or the fixing apparatus in order to support the posture even more. This lumbar support can be reversibly arranged on the support element and/or the fixing apparatus, for example in the form of a pocket into which a support element can be inserted. It is likewise possible for the lumbar support to be secured to the device on the inner or outer side of the support element or the fixing apparatus.

Stiffening elements can be arranged in the support element, the fixing apparatus and/or the tension straps in order to provide additional stability for the wearer of the device. The stiffening elements can be arranged in the support element and also in the fixing apparatus and/or the tension straps, or they can be secured thereto. In one variant of the invention, provision is made for pockets for holding stiffening elements to be formed in the support element and/or the fixing apparatus and/or the tension straps, and so the stiffening elements can easily be inserted into these pockets and removed therefrom when the stiffening elements are no longer required.

Adhesion elements can be arranged on the support element in order to avoid or minimize slippage of the support element on the skin or on the clothing. The adhesion elements can be arranged on the inner side of the support element and/or the fixing apparatus in a punctiform manner or over a large area. The larger the adhesion elements are, the greater the fixing effect is and the smaller the relative movement is between the support element and the body or the clothes.

Provision can furthermore be made for a data logger or a data transmission interface to be connected to the sensor or the sensors in order to record or transmit the data. It is likewise possible for warning signals to be emitted after the data has been evaluated should specific parameters be exceeded. It is likewise possible for the sensors to be used in a bio-feedback method in order to obtain specific feedback, e.g. vibrations, acoustic signals or the like, when specific movements are carried out. The device can also be used in the case of functional electro-stimulation in order to build up or control movements in a targeted manner.

The tension straps are preferably flexible and elastic, wherein provision can be made for a stretch limitation in the tension straps such that, after a specific position is reached or a stretch of the tension straps is reached or exceeded, said tension straps become inelastic, as a result of which there is a perceivable change in the counterforce in the region of the shoulders or the ribcage. There is elasticity in both the extension direction and the bend direction, and so the tension straps can have a design with elasticity in extension and bending.

The support element preferably consists of a shear-stable material such that no relative movement is brought about between the top side and the underside of the support element. The top side faces away from the body of the wearer of the device, while the underside faces the body of the wearer and rests against the skin or the clothes.

In the following text, an exemplary embodiment of the invention is explained in more detail on the basis of the attached figures, in which:

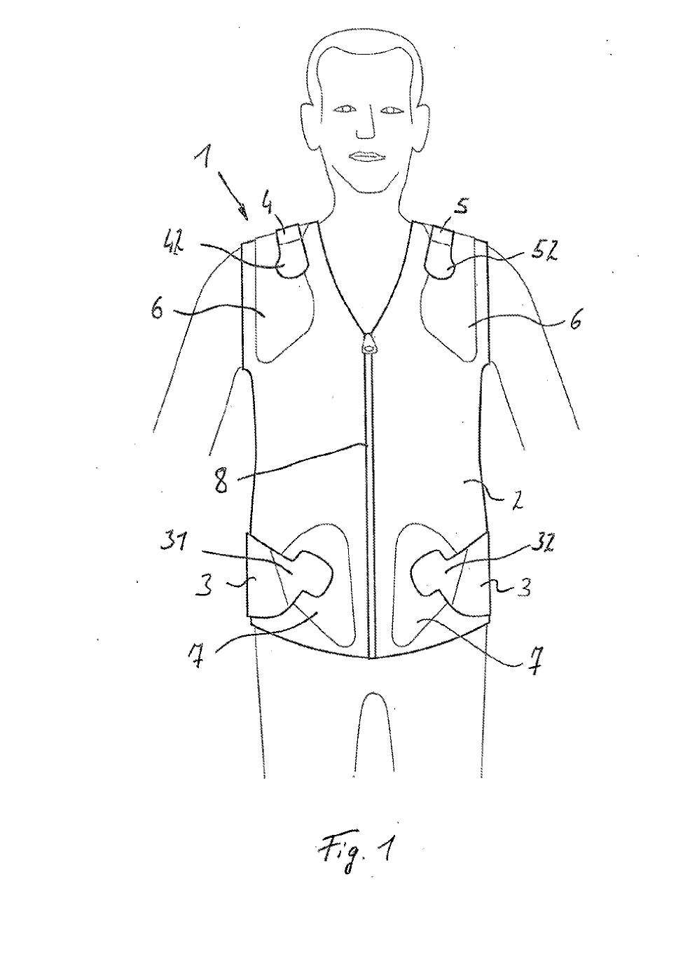

FIG. 1 shows a frontal view of a put-on device;

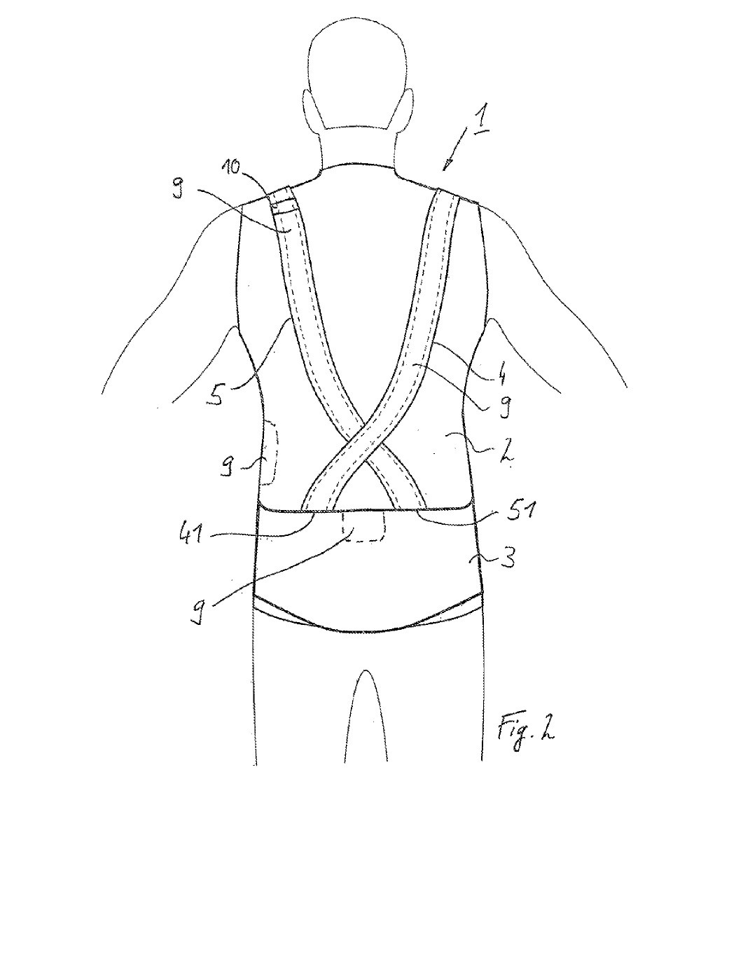

FIG. 2 shows a rear view of a put-on device; and

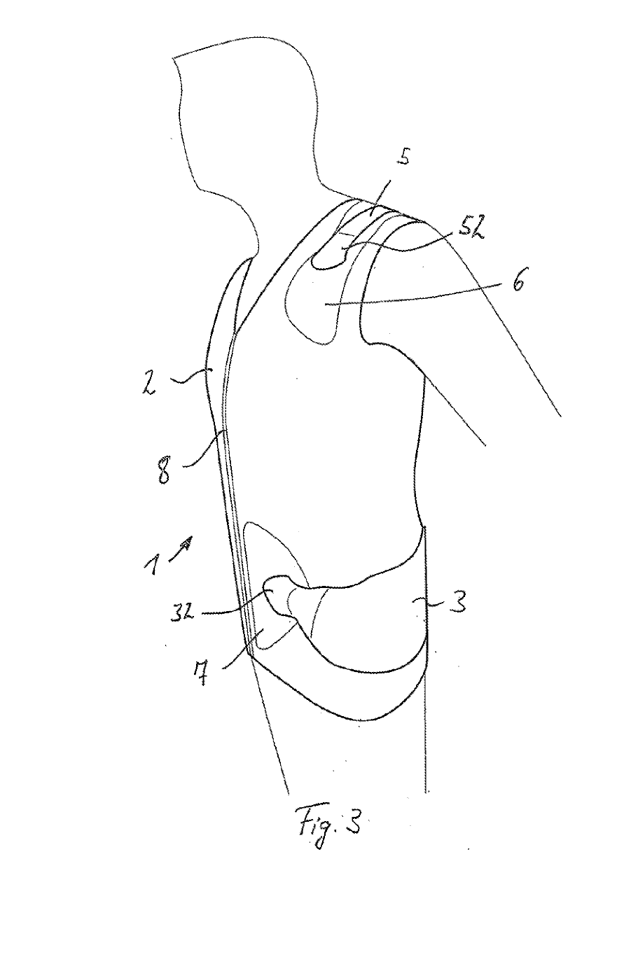

FIG. 3 shows a perspective illustration of a put-on device.

FIG. 1 shows a frontal view of the device 1 for detecting and/or influencing posture, having a flexible support element 2 that is embodied in the form of a vest in the illustrated embodiment. The support element can consist of a textile material, which is preferably provided on the inside thereof with an anti-slip or slip-resistant coating in order to securely hold the support element against the body of the wearer of the device 1. In addition to flexibility, the support element 2 can also have elasticity in order to enable a fit on the body that is as tight as possible. The elasticity may also only be formed in regions in order to improve the fit, whereas the flexible and inelastic regions can be designed for direct force transfer and force transmission. In the front region, the support element 2 has a zip fastener 8 by means of which the support element 2 can be closed after being put on in order to bring about an improved fit and better fit of the support element 2 on the body. Instead of a zip fastener 8, provision can also be made for other fastener apparatuses, for example a hook-and-loop fastener, a so-called touch-and-close fastener, or other connection or fixing apparatuses.

A fixing apparatus 3 is arranged on the support element 2, for example formed thereon or secured thereto, in the region of the waist or the hip. The fixing apparatus 3 is provided as a tension strap, which has a closed embodiment over the rear region of the waist and has two ends 31, 32, which can be displaced with respect to one another, in the frontal region. If the ends 31, 32 are moved toward one another, the fixing apparatus 3 is tensioned, as a result of which a stronger holding force is exerted on the region of the waist. In the illustrated exemplary embodiment, the ends 31, 32 are displaced and fixed via attachment regions 7, which are embodied as part of a hook-and-loop fastener. If hook regions are provided on the inside of the ends 31, 32 of the fixing apparatus 3, the attachment regions 7 are embodied as so-called loop regions, which, in the illustrated exemplary embodiment, extend from the lower edge of the support element 2 to approximately level with the navel on both sides of the zip fastener 8. As a result of embodying the attachment regions 7 and the ends 31, 32 as touch-and-close connections, it is possible to fit the fixing apparatus 3 to the respective wearer in a continuously variable manner.

Arranged in the shoulder and upper chest region of the support element 2 there are attachment apparatuses 6 on which pull elements or tension straps 4, 5 can be fixed. Corresponding to the fixing apparatus 3 of the attachment regions 7, the attachment apparatuses 6 and the ends 42, 52 of the tension straps 4, 5 are embodied as parts of a touch-and-close fastener; the attachment apparatuses 6 are preferably provided as loop regions and the ends 42, 52 of the individual tension straps 4, 5 are provided as hook regions. The positioning of the ends 42, 52 of the tension straps 4, 5 can be selected freely on the attachment apparatuses 6, and so the fine alignment of the ends 42, 52 and the tension in the tension straps 4, 5 can be set individually.

FIG. 2 shows a rear view of the device 1. It is possible to identify in FIG. 2 that the fixing apparatus 3 is arranged in the hip and waist region and has a contiguous design in the dorsal region. To the top, this is adjoined by the material of the support element 2. It can furthermore be identified that the tension straps 4, 5 are designed to cross one another in the region of the back, approximately level with the upper lumbar vertebrae, with the first ends 41, 51 of the tension straps 4, 5 being fixed in the region of the fixing apparatus 3. This fixing can either be permanent by being sewn or welded on, or else it can be reversible by using attachment apparatuses such as touch-and-close fasteners, clasps, webbing loops or buttons. The first ends 41, 51 of the tension straps 4, 5 are arranged on both sides of the spine, that is to say on both sides next to the center line of the support element 2. The distance between the first ends 41, 51 and the center line can be selected relatively freely; the greater the distance is, the greater the effect of a torsional movement is on the second ends 42, 52.

Starting from the attachment sites of the first ends 41, 51, the tension straps 4, 5 are initially guided upward in the direction of the shoulder regions and made to cross in the region of the lumbar vertebrae in the illustrated exemplary embodiment. The further profile of the tension straps 4, 5 extends to the shoulders situated obliquely opposite the attachment sites and said tension straps are fixed to the attachment apparatuses in either the upper shoulder region or in the frontal chest region. The tension straps 4, 5 can have an elastic design over their entire length or only have a partly elastic design over their length. By way of example, a predetermined length of the respective tension straps 4, 5 can have a flexible and inelastic design from the first attachment sites of the first ends 41, 51, whereas a region adjoining thereto, for example in the region of the shoulders, has an elastic design. A stretch limitation can likewise be arranged in the tension straps 4, 5; it becomes effective after a maximum stretch has been reached such that there can be no additional elongation of the tension straps 4, 5 after a predetermined stretch has been reached.

Tension straps 4, 5 can be designed from a single material throughout. It is likewise possible for the tension straps 4, 5 to have different materials and for example to be made of different materials in sections. The tension straps 4, 5 can be embodied as textile straps, ropes, cables, cable pulls or the like. A force coordinate system emerges from the tension straps or pull elements 4, 5 crossing and it can, in particular, detect torsional movements of the wearer of the device and make these perceivable.

Pockets or holding apparatuses 9 for stiffening elements or support elements can be formed in the fixing apparatus 3 and also in the support element 2. A lumbar support can be inserted into a pocket 9 in the region of the central fixing apparatus 3; pockets 9 for stiffening elements can be arranged or formed in the support element 2 in the region of the waist such that this makes it easy to insert cushions, stiffening elements, support apparatuses and the like, and also to remove these again. Instead of pockets 9, it is also possible to provide other holding apparatuses onto which the stiffening elements can be fixed or into which the stiffening elements or support elements can be inserted.

In the illustrated exemplary embodiment, provision is made for a sensor 10 in the tension strap 5 guided over the left shoulder; this sensor can, for example, be embodied as a tensile force sensor. Warning signals can be emitted if a predetermined sensor value is exceeded in order to prevent the wearer of the device 1 from carrying out inexpedient movements. In addition to tensile force sensors, provision can also be made for sensors for accelerations, stretches, pressure, temperature or the like. Provision can likewise be made for conductive textile threads, by means of which sensor signals can be conveyed. The textile threads can be arranged in the straps 4, 5 or in the material of the support element 2. The sensor signals can, in a bio-feedback method, be coupled to vibrators or sound producers or electro-stimulation instruments in order to be able to provide feedback in respect of the current posture or movement. Provision can likewise be made for a data memory or data transmission apparatuses in order to store the sensor data or transmit it to an evaluation unit.

The fixing apparatus 3 is arranged and designed such that the peripheral force exerted by the fixing apparatus 3 is greater than the tensile force of the tension straps 4, 5 in order to prevent a displacement of the support element 2 relative to the wearer. FIG. 3 illustrates a perspective view of the device 1; here, it is possible to identify that the fixing apparatus 3 has an open end 32, which can be fixed to the attachment regions 7 in a reversible and continuously variable manner. As seen in the viewing direction, the second end 52 of the tension strap 5 is fixed in the left shoulder region or in the front left chest region of the wearer, while the first end 51 is, as seen in the viewing direction, arranged to the right of the center line of the support element 2 and hence to the right of the spine of the wearer.

Sensors 10 can also be arranged in stiffening elements and be provided with transmission and/or memory apparatuses in order to document the movements or loads, or to transmit the corresponding data.

As a result of the reversible attachable property of the flexible tension straps or pull apparatuses 4, 5, it is possible to set the tension in the tension straps 4, 5 and make torsional movements perceivable by forces in the region of the shoulders or in the front chest area. It is likewise possible to provide a counterforce against the torsional or bending movement, if desired, in order to prevent twisting of the torso or curving of the spine.

1. A device for detecting and/or influencing posture, having a flexible support element (2), on which a fixing apparatus (3) is arranged for arranging the device (1) at waist height or below, with tension straps (4, 5) being fixed on the fixing apparatus (3) with a first end (41, 51) and guided so as to cross each other in the region of the back and being fixed to the support element (2) in the frontal shoulder or chest region with a second end (42, 52), characterized in that at least one sensor (10) is associated with the support element (2), the tension straps (4, 5) and/or stiffening elements and it detects forces, deformations, angles, temperatures and/or accelerations.

2. The device as claimed in claim 1, characterized in that attachment apparatuses (6) are arranged on the support element (2) in the frontal shoulder or chest region and the second ends (42, 52) of the tension straps (4, 5) can be reversibly fixed to these.

3. The device as claimed in claim 1 or 2, characterized in that the support element (2) is embodied as a jacket or vest.

4. The device as claimed in one of the preceding claims, characterized in that the fixing apparatus (3) is embodied as a tension strap which is part of the support element (2).

5. The device as claimed in one of claims 1 to 3, characterized in that the fixing apparatus (3) and the support element (2) are embodied separately.

6. The device as claimed in one of the preceding claims, characterized in that the fixing apparatus (3) is embodied as a pair of trousers or as a strap in the crotch.

7. The device as claimed in one of the preceding claims, characterized in that a lumbar support is arranged in the support element (2) or in the fixing apparatus (3).

8. The device as claimed in one of the preceding claims, characterized in that stiffening elements are arranged in the support element (2).

9. The device as claimed in one of the preceding claims, characterized in that pockets (9) for holding stiffening or supporting elements are formed in the support element (2) and/or the fixing apparatus (3) and/or in the tension straps (4, 5).

10. The device as claimed in one of the preceding claims, characterized in that adhesion elements are arranged on the inside of the support element (2).

11. The device as claimed in claim 11, characterized in that a data logger or a data transmission interface is connected to the sensor (10).

12. The device as claimed in one of the preceding claims, characterized in that the tension straps (4, 5) are flexible and elastic.

13. The device as claimed in one of the preceding claims, characterized in that the support element (2) is made of a shear-stable material.
